(19) United States
(12) Patent Application Publication
(10) Pub. No.: US 2024/0058209 A1
(43) Pub. Date: Feb. 22, 2024

(54) Boomerang-Type Massage Apparatus

(71) Applicant: STRIG CO., LTD., Seongnam-si (KR)

(72) Inventor: Hwan Kyung OH, Seongnam-si (KR)

(21) Appl. No.: 18/202,610

(22) Filed: May 26, 2023

(30) Foreign Application Priority Data
Aug. 19, 2022 (KR) 10-2022-0104092

(51) Int. Cl. A61H 23/02

(52) U.S. Cl. CPC A61H 23/0254; A61H 2201/0153; A61H 2201/0165

(57) ABSTRACT

Disclosed is a boomerang-type massage apparatus including an inner planar portion, a curved portion extending at an incline from the edge of the inner planar portion, and a blade portion formed so as to be rounded in a thickness direction and to have a counter line curved with various radii of curvature along the edge of the curved portion. The contour line of the blade portion is formed in an elongated S-shape.

This application claims the benefit of Korean Patent Application No. 10-2022-0104092, filed on Aug. 19, 2022, which is hereby incorporated by reference as if fully set forth herein.

### BACKGROUND OF THE INVENTION

### Field of the Invention

The present invention relates to a boomerang-type massage apparatus, and more particularly to a massage apparatus that is formed in a shape of a boomerang including concave portions and convex portions having various curvatures and is capable of generating vibration and microcurrent to massage various parts of the body.

### Description of the Related Art

In general, massage is one of therapies for relieving pain or abnormal phenomena in the body by rubbing skin or muscles or stimulating acupoints to promote blood circulation. Many people use massage therapy to maintain their health.

Massage therapy may be performed manually or using massage instruments. A massagist performs manual massage therapy in a manner of rubbing skin or relaxing muscles of a massage recipient with his/her hands, feet, etc. Such manual massage therapy is advantageous in that a massagist provides a massage recipient with a soft sense of touch and warmth and thus high massage effect is realized. However, such manual massage therapy by a massagist is performed within limited time and place. As massage instruments for massage therapy, electric massage instruments configured to perform rotational or rectilinear reciprocating motion using electrical power, acupressure boards having a plurality of sharp protrusions, and cylindrical manual massage tools are mainly used.

Electric massage instruments using electrical power are convenient to use. However, use of electric massage instruments is significantly limited because the electric massage instruments are high-priced and non-portable due to large sizes thereof and require installation space. Manual massage tools have advantages of low price and high portability and of being used regardless of place and time. However, most manual massage tools have a simple structure including a cylindrical body and a plurality of sharp protrusions formed on the body, and thus provide only an acupressure effect of applying pressure to specific points on the body. If a user rubs his/her body using such a manual massage tool in order to obtain a massage effect, the user may suffer damage to his/her skin due to the sharp protrusions.

Further, conventional massage tools are curved with a constant radius of curvature, and thus it is difficult for a user to massage various parts of the body using a single massage tool.

### SUMMARY OF THE INVENTION

The present invention has been made to solve the above problems, and it is an object of the present invention to provide a massage apparatus that is formed in a shape of a boomerang including concave portions and convex portions having various curvatures and is capable of generating vibration and microcurrent to massage various parts of the body.

In accordance with the present invention, the above and other objects can be accomplished by the provision of a boomerang-type massage apparatus including an inner planar portion, a curved portion extending at an incline from the edge of the inner planar portion, and a blade portion formed so as to be rounded in a thickness direction and to have a counter line curved with various radii of curvature along the edge of the curved portion, wherein the contour line of the blade portion is formed in an elongated S-shape.

The blade portion may include a single-edged blade portion including an inclined curved surface formed on the front portion of the massage apparatus and a double-edged blade portion including inclined curved surfaces formed on the front portion and the rear portion of the massage apparatus.

The inner planar portion may include a front planar portion and a rear planar portion, and the massage apparatus may further include a front grip cover coupled to the front planar portion and a rear grip cover coupled to the rear planar portion.

The massage apparatus may further include a printed circuit board configured to control operation of the massage apparatus and to generate microcurrent, a rechargeable battery, and a vibration motor configured to generate vibration. The printed circuit board, the battery, and the vibration motor may be mounted in the massage apparatus so as to be located between the front grip cover and the rear grip cover.

An electrode part configured to cause the microcurrent to flow to a user's body when the user's body touches the electrode part and a power button may be disposed on the front grip cover, and a charging terminal may be disposed on the rear grip cover.

### BRIEF DESCRIPTION OF THE DRAWINGS

The above and other objects, features, and other advantages of the present invention will be more clearly understood from the following detailed description taken in conjunction with the accompanying drawings, in which:

FIG. 1 is a front perspective view showing a boomerang-type massage apparatus according to an embodiment of the present invention.

FIG. 2 is a rear perspective view showing the boomerang-type massage apparatus according to the embodiment of the present invention.

FIG. 3 is a perspective view showing a state in which the massage apparatus according to the embodiment of the present invention is placed in a charging cradle.

FIG. 4 is a partial perspective view showing a power button and a charging terminal of the massage apparatus according to the embodiment of the present invention; and

FIG. 5 is a view showing the internal structure of the massage apparatus according to the embodiment of the present invention.

### DETAILED DESCRIPTION OF THE INVENTION

The present invention will now be described more fully hereinafter with reference to the accompanying drawings, in which various embodiments are shown. The examples, however, may be embodied in many different forms and should not be construed as being limited to the embodiments set forth herein. It is to be understood that the present invention covers all modifications, equivalents, and alternatives falling within the scope and spirit of the present invention.

The terminology used herein is for the purpose of describing particular embodiments only and is not intended to be limiting of exemplary embodiments of the invention. As used herein, the singular forms are intended to include the plural forms as well, unless the context clearly indicates otherwise. It will be further understood that the term “include” or “have”, when used herein, specifies the presence of stated features, integers, steps, operations, elements, components, or combinations thereof, but do not preclude the presence or addition of one or more other features, integers, steps, operations, elements, components, or combinations thereof.

Hereinafter, exemplary embodiments of the present invention will be described in detail with reference to the accompanying drawings. Wherever possible, the same reference numerals will be used throughout the drawings to refer to the same or like parts. In the following description of the present invention, a detailed description of known functions or known configurations incorporated herein will be omitted when it may make the subject matter of the present invention rather unclear. In the drawings, elements may be exaggerated in size, omitted, or schematically illustrated for convenience in description and clarity.

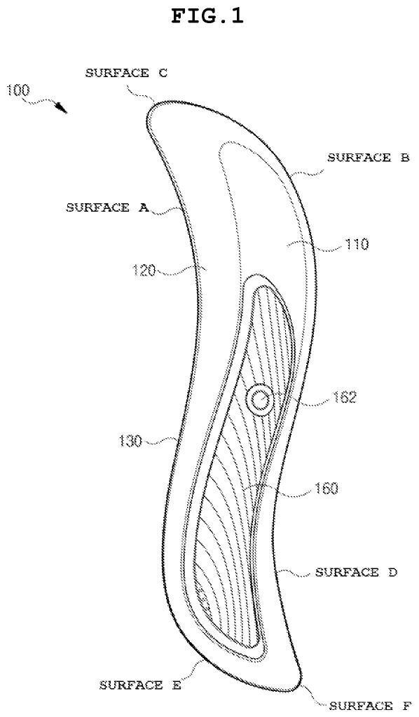

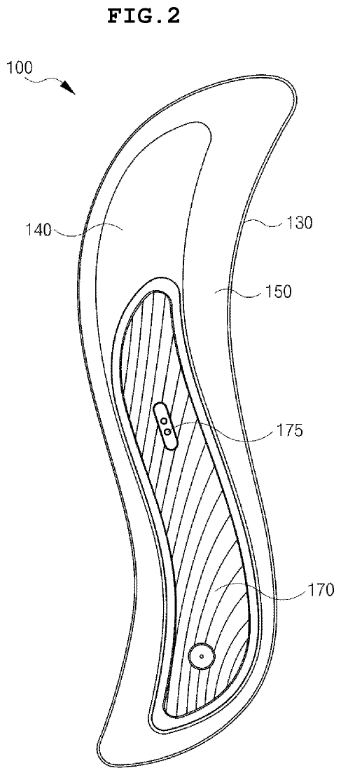

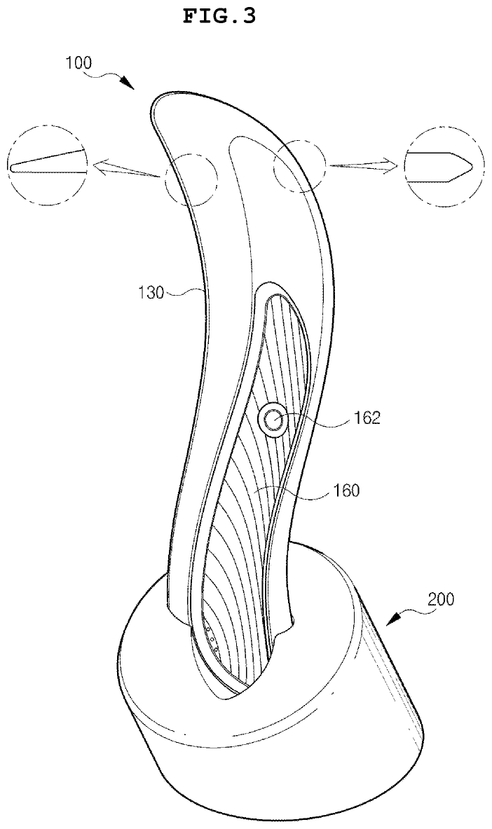

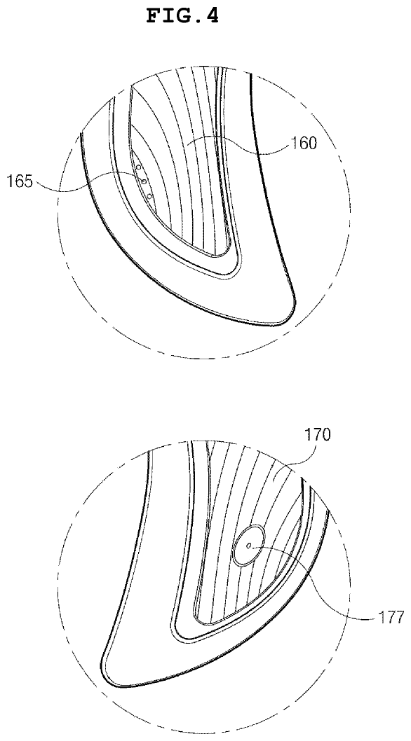

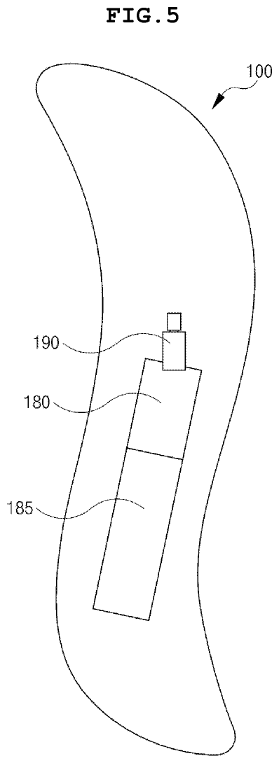

FIG. 1 is a front perspective view showing a boomerang-type massage apparatus according to an embodiment of the present invention, FIG. 2 is a rear perspective view showing the boomerang-type massage apparatus according to the embodiment of the present invention, FIG. 3 is a perspective view showing a state in which the massage apparatus according to the embodiment of the present invention is placed in a charging cradle, FIG. 4 is a partial perspective view showing a power button and a charging terminal of the massage apparatus according to the embodiment of the present invention, and FIG. 5 is a view showing the internal structure of the massage apparatus according to the embodiment of the present invention.

The boomerang-type massage apparatus 100 according to the present invention includes inner planar portions 110 and 140, curved portions 120 and 150 extending at an incline from the edges of the inner planar portions 110 and 140, and a blade portion 130 formed so as to be rounded in a thickness direction and to have a counter line curved with various radii of curvature along an edge defined by the curved portions 120 and 150.

The massage apparatus 100 may be formed such that the contour line of the blade portion 130 has an elongated S-shape. Referring to FIG. 1, the upper end portion and the lower end portion of the massage apparatus 100 may be formed such that the contour lines thereof are rounded, rather than being sharp. On the whole, the massage apparatus 100 may be formed asymmetrically with respect to a virtual center thereof.

As shown in FIGS. 1 and 2, the inner planar portions 110 and 140 may include a front planar portion 110 defining an inner portion of the front surface of the massage apparatus 100 and a rear planar portion 140 defining an inner portion of the rear surface of the massage apparatus 100. The front planar portion 110 and the rear planar portion 140 may be formed parallel to each other.

A front curved portion 120 may be formed along the edge of the front planar portion 110 so as to extend at an incline from the edge of the front planar portion 110, and a rear curved portion 150 may be formed along the edge of the rear planar portion 140 so as to extend at an incline from the edge of the rear planar portion 140. Each of the curved portions 120 and 150 may be formed so as to surround a corresponding one of the inner planar portions 110 and 140 and to have different inclination angles at different points.

The blade portion 130 may be formed so as to be rounded in the thickness direction along an edge where the front and rear curved portions 120 and 150 meet each other. The blade portion 130 may be formed such that the contour thereof includes concave surfaces and convex surfaces having different radii of curvature.

As shown in FIG. 1, the blade portion 130 may be divided into about six portions although the boundaries between the six portions do not appear clearly. In detail, based on FIG. 1, the blade portion 130 may include a concave surface A corresponding to the left-upper portion thereof, a convex surface B corresponding to the right-upper portion thereof, a surface C corresponding to the upper end portion thereof, a concave surface D corresponding to the right-lower portion thereof, a convex surface E corresponding to the left-lower portion thereof, and a surface F corresponding to the lower end portion thereof. The surface A may have a slightly larger radius of curvature than the surface D, and the surface B may have a slightly larger radius of curvature than the surface E. Since the blade portion 130 includes the concave surfaces and the convex surfaces having different radii of curvature at different points, the concave surfaces and the convex surfaces of the blade portion 130 may be selectively used for massage depending on the area or the curved shape of a part of the body to be massaged.

One side of the blade portion 130 may be formed as a single-edged blade including only a front inclined curved surface, and the opposite side of the blade portion 130 may be formed as a double-edged blade including a front inclined curved surface and a rear inclined curved surface.

As shown in FIG. 3, the single-edged blade refers to a blade in which an inclined curved surface extending to the edge of the blade portion 130 is formed only on the front portion of the massage apparatus, and the double-edged blade refers to a blade in which an inclined curved surface extending to the edge of the blade portion 130 is formed on the rear portion of the massage apparatus as well as on the front portion of the massage apparatus.

In the case of a single-edged blade, because the entire area of one surface thereof touches skin, it is possible to apply relatively high pressure to specific points on the body. Therefore, physical therapists mainly use a single-edged blade. On the other hand, in the case of a double-edged blade, it is not suitable for application of high pressure, and thus it is difficult to stimulate points deep in the muscle. However, it is suitable for low-pressure massage of stimulus-sensitive parts of the body, and therefore, it is appropriate for ordinary users. In the massage apparatus 100 according to the present invention, since one side of the blade portion 130 is formed as a single-edged blade and the opposite side of the blade portion 130 is formed as a double-edged blade, it is possible for a user to selectively use one of the two types of blades as needed.

Referring to FIG. 1, the surfaces C, A, and E of the blade portion 130 may be formed as single-edged blades, and the surfaces B, D, and F of the blade portion 130 may be formed as double-edged blades. In particular, the surface A may be used for massage of body parts having a relatively large area, such as a neck, shoulders, flanks, and thighs, and the surface B may be used for massage of body parts having a relatively small area, such as arms.

Generally, the surface A may be used most frequently, and the frequency of use may gradually decrease in the order of the surface B, the surface C, the surface D, the surface E, and the surface F.

As shown in FIGS. 1 and 2, the massage apparatus 100 according to the present invention may further include a front grip cover 160 coupled to the front planar portion 110 and a rear grip cover 170 coupled to the rear planar portion 140.

The front grip cover 160 may be coupled to a cut-out portion formed in the lower portion of the front planar portion 110, and the rear grip cover 170 may be coupled to a cut-out portion formed in the lower portion of the rear planar portion 140. The massage apparatus 100 may further include an inner space defined in the main body thereof in order to accommodate electronic components between the front grip cover 160 and the rear grip cover 170.

Each of the front grip cover 160 and the rear grip cover 170 may have a plurality of curved grooves formed in the surface thereof so that a user easily holds the massage apparatus 100.

The planar portions 110 and 140 and the curved portions 120 and 150 of the massage apparatus 100 may be made of stainless steel, and the front grip cover 160 and the rear grip cover 170 may be made of plastic, which is an elastic non-conductive material having a certain degree of friction.

As shown in FIG. 5, the massage apparatus 100 according to the present invention may further include a printed circuit board (PCB) 180 configured to control operation of the massage apparatus 100 and to cause microcurrent to flow, a rechargeable battery 185, and a vibration motor 190 configured to generate vibration. The PCB 180, the battery 185, and the vibration motor 190 may be mounted in the massage apparatus 100 so as to be located between the front grip cover 160 and the rear grip cover 170.

The PCB 180 may be formed in a substantially rectangular shape, and may be mounted in the inner space defined in the massage apparatus 100 so as to be located between the front grip cover 160 and the rear grip cover 170. The PCB 180 may serve to control operation of the massage apparatus 100 and to flow microcurrent.

The battery 185 may be mounted in the inner space defined in the massage apparatus 100 so as to be located between the front grip cover 160 and the rear grip cover 170. The battery 185 may be implemented as a secondary battery such as a lithium-ion battery, and thus may be rechargeable.

As shown in FIG. 5, The vibration motor 190 may be mounted in the inner space defined in the massage apparatus 100, and may be connected to a top side of the PCB 180. The vibration motor 190 may be provided at an end portion of the rotary shaft thereof with a weight in order to generate vibration of 60 Hz. The PCB 180 may serve to control the vibration motor 190 in order to adjust the intensity of vibration, and may also serve to control the vibration motor 190 to generate vibration such that the intensity of vibration changes periodically.

As shown in FIGS. 1, 3, and 4, the massage apparatus 100 according to the present invention may further include an electrode part 162, which is configured to cause microcurrent to flow to a user's body when the user's body touches the electrode part 162, and a power button 165. The electrode part 162 and the power button 165 may be disposed on the front grip cover 160.

When a part of the user's body (e.g. a hand) touches the electrode part 162, the electrode part 162 may generate microcurrent to flow to the user's body through the blade portion 130. In detail, when a user's finger or a part of the user's body to be massaged touches the electrode part 162, the PCB 180 may generate microcurrent, and the microcurrent may flow to a part of the user's body through the blade portion 130, whereby massage using microcurrent may be realized.

Based on FIG. 4, the power button 165 may be disposed on a lower portion of the front grip cover 160. The power button 165 may serve to turn the massage apparatus 100 on/off. In addition, the power button 165 may include a switch for control of the intensity of microcurrent and a switch for control of the intensity of vibration.

As shown in FIG. 4, the massage apparatus 100 according to the present invention may further include a charging terminal 177. Based on FIG. 4, the charging terminal 177 may be disposed on a lower portion of the rear grip cover 170. As shown in FIG. 3, when the massage apparatus 100 is placed in a charging cradle 200, the charging terminal 177 may be electrically connected to a terminal of the charging cradle 200, and accordingly, the battery 185 may be charged. In addition, the charging terminal 177 may be configured to allow a separate charging cable to be directly inserted thereinto to be electrically connected thereto.

In addition, as shown in FIG. 2, the massage apparatus 100 according to the present invention may further include a charge state indicator 175. Based on FIG. 2, the charge state indicator 175 may be disposed on an upper portion of the rear grip cover 170. The charge state indicator 175 may include a plurality of light-emitting diodes (LEDs) in order to indicate the state of charge of the battery 185. In this case, the number of LEDs turned on or the colors of the LEDs may change depending on the state of charge of the battery 185.

As is apparent from the above description, the boomerang-type massage apparatus according to the present invention may be formed in a shape of a boomerang including concave portions and convex portions having various curvatures, and may generate vibration and microcurrent, thereby easily and effectively massaging various parts of the body.

Although specific embodiments of the present invention have been disclosed for illustrative purposes, those skilled in the art will appreciate that various modifications, additions, and substitutions are possible, without departing from the scope and spirit of the invention as disclosed in the accompanying claims. Further, such modifications, additions, and substitutions of the present invention are to be appreciated as being included in the scope and spirit of the invention.

What is claimed is:

1. A boomerang-type massage apparatus comprising:
an inner planar portion;
a curved portion extending at an incline from an edge of the inner planar portion; and
a blade portion formed so as to be rounded in a thickness direction and to have a counter line curved with various radii of curvature along an edge of the curved portion,
wherein the contour line of the blade portion is formed in an elongated S-shape.

2. The boomerang-type massage apparatus according to claim 1, wherein the blade portion comprises:
a single-edged blade portion comprising an inclined curved surface formed on a front portion of the massage apparatus; and
a double-edged blade portion comprising inclined curved surfaces formed on the front portion and a rear portion of the massage apparatus.

3. The boomerang-type massage apparatus according to claim 1, wherein the inner planar portion comprises a front planar portion and a rear planar portion, and
wherein the massage apparatus further comprises:
a front grip cover coupled to the front planar portion; and
a rear grip cover coupled to the rear planar portion.

4. The boomerang-type massage apparatus according to claim 3, further comprising:
a printed circuit board configured to control operation of the massage apparatus and to generate microcurrent; and
a vibration motor configured to generate vibration,
wherein the printed circuit board and the vibration motor are mounted in the massage apparatus so as to be located between the front grip cover and the rear grip cover.

5. The boomerang-type massage apparatus according to claim 4, further comprising:
an electrode part configured to cause the microcurrent to flow to a user's body when the user's body touches the electrode part; and
a power button,
wherein the electrode part and the power button are disposed on the front grip cover.

6. The boomerang-type massage apparatus according to claim 4, further comprising a charging terminal disposed on the rear grip cover.

7. The boomerang-type massage apparatus according to claim 3, further comprising a rechargeable battery mounted in the massage apparatus so as to be located between the front grip cover and the rear grip cover.
